(19) United States
(12) Patent Application Publication
(10) Pub. No.: US 2008/0294071 A1
(43) Pub. Date: Nov. 27, 2008

(54) Assay Method

(76) Inventors: Laura Corradini, Kent (GB); Mark John Field, Kent (GB)

Correspondence Address: PFIZER INC., PATENT DEPARTMENT, MS8260-1611, GROTON, CT, 06340, US

(21) Appl. No.: 11/569,328

(22) PCT Filed: May 10, 2005
(86) PCT No.: PCT/IB2005/001387

§ 371 Date: Mar. 4, 2008

Related U.S. Application Data
(60) Provisional application No. 60/588,221, filed on Jul. 14, 2004.

(30) Foreign Application Priority Data
May 19, 2004 (GB) 0411141.5

(51) Int. Cl. A61B 5/11

(52) U.S. Cl. 600/595; 128/898

(57) ABSTRACT

An assay for the assessment of drug side effects particularly the side effect of dizziness comprising the steps of: providing a first control animal located on abeam; inducing the control animal to traverse the beam; recording the number of footslips made by the animal during the traversal; providing a second test animal located on the beam or duplicate beam; inducing the test animal to traverse the beam; recording the number of footslips made by the animal during the traversal and determining whether there is an increase, decrease or no change in the number of footslips made by the test animal in comparison to the control animal.

This invention relates to a pre-clinical animal model for the assessment of drug side effects particularly the side effect of dizziness.

### BACKGROUND TO THE INVENTION

It is commonly appreciated that many clinically effective compounds used in the treatment of human medical conditions possess side effect activities in addition to their medically relevant activity. For example in addition to their analgesic effect the compounds morphine and tramadol are known to produce the side effects including somnolence (characterised by tendency to fall asleep), mental confusion, drowsiness and sedation (characterised by a calming of nervous excitement or decreased alertness and induction of a state of rest or sleep). The degree to which compound related side effects are manifested in the dosed subject often vary with the amount of compound dosed and with the progression of time since administration of the dose.

Some compound side effects have a more pronounced effect on the quality of life of the dosed subject than others, of particular concern are side effects related to the central nervous system (CNS). Dizziness for example can prevent a subject from performing coordinated locomotor tasks ranging from inhibiting the safe performance of gross movements requiring co-ordination, such as walking or manual labour, and additionally from co-ordination of fine motor skills such as are required when driving a car or climbing stairs or handling objects. The state of dizziness is complex and includes faintness, giddiness, light headedness and unsteadiness, it may be linked to a disturbance in the vestibular sense and vestibulomotor function leading to a deficiency in fine motor co-ordination. Dizziness can also produce nausea and a feeling of sickness sometimes leading to vomiting.

The ideal drug or pharmaceutical compound would demonstrate clinical efficacy for a given medical condition without any associated side effects, particularly CNS effects. It is desirable therefore to be able to test for such compound associated side effects at as early a stage as possible, preferably before the compound enters into human clinical trials. Consequently it is desirable to provide a preclinical animal model that can detect and quantify a clinically relevant side effect generally recorded in humans in the clinic using subject questionnaires and verbal i visual rating scales.

Animal models have been developed and used for a small variety of CNS related side effects of compounds, for example the locomotor test which can provide a measure of sedation, ataxia (characterised by muscle relaxation or lack of muscle tone) or catalepsy. However no measure for the side effect of dizziness has yet been demonstrated.

Dizziness is a particularly difficult side effect to examine in an animal. Unlike many side effects dizziness is not readily measured from mere observation. For example observation of the general locomotion of an animal can determine catalepsy (a trance like state with loss of voluntary motion or rigid maintenance of a body position over an extended period of time) as indicated by a decrease from normal in the general locomotion and exploratory behaviour of the animal over a period of time, or ataxia (muscle relaxation or lack of muscle tone, leading to co-ordination failure) as indicated by a decrease from normal in the number of instances of the animal rearing on the hind legs. A specific experimental test to indicate whether an animal is experiencing dizziness would be very advantageous and before the present invention this has been unavailable.

We have demonstrated that a beam walking method can be used to measure drug-induced dizziness in an animal subject and that the results obtained correlate well with those recorded in the clinic. Furthermore the data from the beam walking method can be used to distinguish compounds known to cause dizziness in the clinic from compounds causing somnolence, hypnosis, sedation, ataxia or pychostimulant effects.

Beam walking methods are known and have been used to gauge the degree of brain damage in animals post physical trauma. The existing methods involve measurements, from animals that are physically impaired in motor regions of the brain, such as recording the time taken for the animal to cross the beam or merely placing the animal on the beam to record whether the animal would remain in place or fall (Feeney D M, Gonzalez A, Law W A, Science. 1982; 217:855-857. Goldstein L B, Davis J N, Behav Neurosci. 1990; 104:318-325). Neither of these existing measurements provide a measure of dizziness, which is more reasonably measured in animals not effected by physical damage to motor regions of the CNS (that is brain damaged animals). However we have determined that the measurement of a new and different variable, the number of foot slips performed by an animal during crossing the beam in a beam walking method, does measure this dizziness effect, thus providing a balance and co-ordination endpoint measure.

### BRIEF DESCRIPTION OF THE INVENTION

The invention makes available a method of assessing the degree to which an animal experiences dizziness. The invention permits the identification of compounds that induce dizziness as a side effect. The advantage of the method is that it allows the assessment of dizziness in an animal as distinguished from other common CNS effects such as somnolence, sedation, ataxia or psycho stimulant effects, this measure of dizziness also correlates well with results from equivalent clinical methods.

### DETAILED DESCRIPTION OF THE INVENTION

According to a first aspect of the present invention there is provided a method for determining the degree to which an animal experiences dizziness comprising the following steps:
a) providing a first “control” animal located on a narrow, raised, length of beam,
b) inducing the control animal to traverse the beam,
c) recording the number of footslips made by the animal during the traversal, wherein a footslip is the misplacing of any foot of the animal in the process of taking a step such that the foot in the process of performing a step does not contact the beam or contacts the beam but falls away or on contact is adjusted or replaced before successfully bearing the weight of the animal, or causes the animal to fall,
d) separately providing a second “test” animal located on the narrow raised length of beam or duplicate beam,
e) inducing the test animal to traverse across the beam,
f) recording the number of footslips made by the animal during the traversal,
g) determining whether there is an increase, decrease or no change in the number of footslips made by the test animal in comparison to the control animal.

According to a second aspect of the invention there is provided a method for assaying a compound for the effect of producing dizziness in animal comprising the following steps:
a) providing a “control” animal located on a narrow, raised, length of beam,
b) inducing the control animal to traverse the beam,
c) recording the number of footslips made by the animal during the traversal, wherein a footslip is the misplacing of any foot of the animal in the process of taking a step such that the foot in the process of performing a step does not contact the beam or contacts the beam but falls away or on contact is adjusted or replaced before successfully bearing the weight of the animal or causing the animal to fall from the beam,
d) providing a “test” animal which has been dosed with a test compound, a) providing the dosed “test” animal located on the narrow raised length of beam or duplicate beam,
f) inducing the test animal to traverse the beam
g) recording the number of footslips made by the animal during the traversal,
h) determining whether there is an increase, decrease or no change in the number of footslips made by the test animal in comparison to the control animal.

The animal is preferably a non human animal and may be any member of the animal kingdom possessing limbs and capable of locomotion using the limbs in a stepwise fashion on a surface. The animal is preferably a mammal, more preferably a rodent, further preferably a rat or a mouse, most preferably a rat.

The “control” animal, can be a representative normal healthy animal of it's class not suffering from obvious physical impairment, particularly impairment to the CNS for example due to neurological disease. The control animal is preferably selected on the criterion of traversing the beam without pausing or freezing in motion across the beam. More preferably the control animal should demonstrate no more than two footslips over the distance of traversal. The control animal may additionally be treated with a vehicle, i.e. treated with the solution in which an active compound would be delivered to the test animal but lacking the active compound, essentially a placebo dose.

Preferably the animal is located on the beam towards one end of the beam.

More than one control animal may be used for the purposes of the performance of the method in order to gain statistical confidence in the measure of footslips for a representative control animal.

There are numerous methods of inducing the animal to cross the beam, for example the animal may be caused to move from a region of a negative or aversive stimulus, or caused to move from a region of a negative aversive stimulus towards a region of a positive or rewarding stimulus. Examples of a negative or aversive stimulus can include the presence of noise, bright light, cold temperature, delivery of a pain stimulus and examples of a positive or rewarding stimulus can include the presence of quietness, darkness, warmth, food, water alcohol, sugar, the animals own home cage or dwelling, presence of pups, progeny or a mate or animal of the opposite sex, the inclination of the beam may also give an incentive for the animal to cross. Preferably the animal is induced to cross the beam by providing a bright light in the region of the beam at the beginning of the traversal and darkness at the opposite end of the beam where the traversal ends.

The animal can be induced to traverse all the way or part of the way across the beam, preferably the animal is induced to traverse all the way across the beam.

The measurement of the number of footslips may by an animal be made over the distance of the full length or partial length of the beam. Preferably the measurement of the number of footslips is made over a constant distance of traversal of the beam by the animal in the performance of any one method, most preferably over the distance of the entire length of the beam. Most preferably, footslip measurement comparisons are made between animals that have traversed the same distance on the beam.

The beam is preferably longer than the longitudinal length of the animal in order to allow the animal a reasonable distance over which to traverse and is more preferably several times longer than the body length of the animal subject (for example the number of steps to traverse the beam may be in the region of 10-20). The beam is preferably a long, narrow, straight, strip of material (for example a solid rod, pole, plank or a taut rope or wire) capable of supporting the weight of the animal without significant deformation and is positioned with its longitudinal aspect essentially parallel to but at a distance from the ground, however the aspect of the beam may be inclined to the ground if required. The beam is preferably narrower than the transverse width of the animal but wider than its individual foot width of the animal, more preferably the beam has a width of between 1 to 10 times the width of the animals foot, most preferably between 1 to 3 times the width of the animals foot. Preferably the beam has an essentially flat and planar surface on which the animal can walk.

It is important to train animals in beam walking prior to their use as test or control animals. Training usually takes place over a series of days during which an animal is initially given a short distance to traverse the beam and is allowed to repeat the traversal during which time the distance traversed in gradually increased to that to be used during the experimental measurements. Animals intended to be used as control or test animals (for example prior to dosing with compound or any other intervention, surgery or treatment) are excluded from future use in the assay if they fail to cross the beam due to falling, pausing, or freezing during the traversal, not moving from the start of the traversal, performing more than two footslips in a traversal.

The term footslip is considered to be a misplacing of any foot of the animal in the process of taking a step such that the foot in the process of performing a step does not contact the beam or contacts the beam but falls away or on contact is adjusted or replaced before successfully bearing the weight of the animal. It also includes the misuse of a foot in raising it and relying on other feet to substitute its place in a step, for example in the process of jumping or hopping forward. Any foot may be monitored in order to provide a record of a footslip; preferably a rear foot or hind paw is monitored.

The test animal of aspect 1 may be the same as the control animal or may be a different animal but of the same class. The test animal may be a normal or healthy animal or may possess a condition which might possibly promote dizziness, for example a condition due to damage to or alteration of the CNS caused by for example, trauma, operative procedure, disease, pathogenic infection, contact with a chemical or biological substance or with radiation, genetic phenotype, genetic modification, metabolic or hormonal imbalance or change; the test animal may also have been treated with a pharmacological active compound, which may potentially induce dizziness.

The test animal of aspect 2 may be the same as the control animal or may be a different animal but of the same class. Preferably the test animal prior to dosing with the test compound is a representative normal healthy animal of it's class not suffering from obvious physical impairment of CNS/neurological disease and is selected on the criterion of traversing the beam without falling, remaining stationary from the outset of the test, pausing or freezing in motion across the beam. More preferably the test animal prior to dosing with the test compound should demonstrate no more than two footslips over the distance of traversal.

The dizziness side effect produced by a compound can be ranked according to the degree to which there is a measured increase or decrease in the number of footslips made by the dosed test animal in comparison to the control animal, thus various test compounds can be ranked with respect to the control and with respect to each other in degree of effect produced.

More than one test animal may be used for the purposes of the performance of the method in order to gain statistical confidence in the observations measured.

In a further embodiment of either the first or second aspect of the invention the time taken to make the traversal can also be recorded and it can be determined whether there is an increase, decrease or no change in the time to make the traversal by the test animal in comparison to the control animal.

According to a third aspect of the present invention there is provided the method according to either aspect 1 or aspect 2 further comprising the steps of
a) performing a second different test designed to measure the degree of locomotor activity for the test animal and control animal
b) determining whether there is an increase, decrease or no change in the degree of locomotor activity measured for the test animal in comparison to the control animal.

The measured locomotor activity may be vertical and/or horizontal locomotion, latency to fall from a rota rod, time to cross a raised beam, number of entries into a region of an open arena, preferably the measured locomotor activity is vertical and/or horizontal locomotion

The second test may be a different method that can be used to measure locomotor activity or motor co-ordination such as tests known in the art, preferably the rotarod test (Jones, B. J. and Roberts, D. J. (1968): Naunun-Schmeidebergs Archives of Pharmacology 259: 211), the open field test (Prut L and Beizung, C., Eur J. Pharmacol. 2003; 463::3-33), the locomotor activity test (Salmi P and Ahlenius S., Neuroreport. 2000 Apr. 27; 11 (6):1269-72), most preferably the locomotor test.

For example the locomotor activity test can be used to measure and compare a control animal with a test animal by collecting comparative data for horizontal activity (locomotor activity including total distance covered (cm) in a period, and centre distance (cm) the centre distance can be divided by the total distance to obtain a centre distance to total distance ratio), vertical activity (number of instances in a period of rearing to balance on the hind limbs for example in the process of standing reaching or leaping or jumping or climbing), For example such data can be collected in 2 to 5 minute intervals over a 30-minute test session for the control and test animal. The control and test animals can be the same or equivalent animals to those used in either the first or second aspect of the invention.

The locomotor activity test can be performed by recording the spontaneous locomotor activity of animals, for example rats, in a novel environment. The test arena is equipped with photocells located at a suitable distance above floor level to allow the recording of horizontal and vertical activity, approximately 2 and 15 cm above the floor for rats (San Diego Instruments, CA, USA). Each animal is placed in the centre of the area and the total locomotor activity (horizontal and vertical) is monitored for example every 5 min for a maximal time period of 30 min for control and test animals. A decrease in the degree of horizontal locomotion of the test animal with respect to the control can be indicative of catalepsy, sedation, hypnosis, or somnolence. An increase in the degree of horizontal locomotion (for example the horizontal distance covered by an animal in a time period in the process of walking or running normally) of the test animal with respect to the control can be indicative of psychomotor stimulation and hyperactivity. Likewise a decrease in the degree of vertical locomotion (for example the number of times an animal rears on its hind legs in a time period) only of the test animal with respect to the control can be indicative of ataxia. An increase in the degree of vertical locomotion of the test animal with respect to the control can be indicative of psychomotor stimulation and hyperactivity. Thus the combination of either the first or second aspect of the invention with the further performance of a second method, preferably a locomotor activity test, can be used to determine the presence or absence of any other closely related motor coordination or locomotion effect.

In a further embodiment of any one of the first, second or third aspects of the invention more than one control and/or test animal may be used.

The term “test compound” as used herein is intended to include pharmaceutical compounds and drugs.

The test compound can be delivered by any standard method for example orally or intravenously or intraperitoneal injection or injected intramuscularly or injected subcutaneously or by inhalation or by suppository or pessary or topically, preferably the dose is delivered orally. The dose of a compound is typically of the range from 0.01 to 1000 mg/kg body weight of the subject animal, preferably 0.1 to 100 mg/kg. Alternatively the dose may be delivered by intravenous infusion, preferably at a dose which of the range from 0.001-1000 mg/kg/hr, more preferably at a dose which of the range from 0.001-1000 mg/kg/hr. The above dosages are exemplary of the average case and may be more or less in quantity accordingly.

In modification of the first, second or third aspect of the invention more than one test compound may be administered.

The following examples illustrate the embodiments and principles of the invention.

### EXAMPLES

### Methods

### Animals

Male Sprague Dawley rats 200-300 g (Charles River, Margate, U.K.) were housed in group of five per cage under a 12 h light/dark cycle with food and water available ad libitum. Each experiment was carried out with groups of at minimum 7 rats. All procedures in this study were performed in accordance with the Home Office Animals (Scientific Procedures) Act 1986 and accordingly with our Project License, after the experiment, animals were sacrificed by schedule 1 method.

### Locomotor Activity Test

The spontaneous locomotor activity of rats in a novel environment was monitored for 30 min in a 35×20 cm Perspex chamber. The cage was equipped with two series photocells located at 2 and 15 cm above the floor (San Diego Instruments, CA, USA). To measure drug-induced decrease locomotion such as morphine and gabapentin, at a pre-defined time post drug administration, each animal was placed in the centre of the cage. To measure drug-induced increase in locomotion such as phencyclidine (PCP) rats were placed in the cage at least 30 min before recording. total activity (ambulation and rearing) was monitored every 5 min for 30 min.

### Beam Walking Test

The Beam walking apparatus consists of a 1.5 m long beam with a 2.5×2.5 cm square cross section, elevated 75 cm above the floor. The test was performed in dim light conditions (18 lux). A light source (520 lux) was placed at the start-end of the beam while a dark box at the other side (Feeney D M et al, Amphetamine, Haloperidol, and experience interact to affect rate of recovery after motor cortex injury, Science 217, 1982). Rats were habituated to the dim light condition for at least 1 hour before the beginning of the training sessions. Rats were trained to cross the beam over 2 days, twice a day. The first day, rodents were trained to cross starting from last quarter and half of the beam until the dark box, in the first and second session, respectively. The following day rodents were trained to cross the entire length of the beam twice. At least 2 hours were left between each daily session. On the day of test a baseline recording was registered before compound administration and rats were selected based on their ability to cross the beam with no major impairments. Therefore, only rodents that crossed the beam in less than 10 seconds and showing two or less foot slips were used for the assessment of drug-induced motor impairments. Then rats were tested for their ability to cross the beam at various time points after drug injection. The time taken to cross and the number of foot slips produced while a rat was crossing the beam were counted. A maximum cut off score of 30 seconds and 5 foot slips, respectively was given to those rats that did not cross or fell off the beam. No movement or freezing behaviours were also scored with the maximum value.

### Test Compounds

Morphine sulphate (1, 3 and 10 mg/kg, sc) and phencyclidine (PCP; 0.1-1-10 mg/kg, ip) were supplied by Sigma Aldrich (Gillingham, UK) and dissolved in physiological saline. Gabapentin (30, 100 and 300 mg/kg, PO) was synthesis in house (Pfizer Lab, Ann Arbor, Mich., USA) and dissolved in water.

### Data Analysis

In the locomotor activity task, total counts are the sum of horizontal and vertical movements (photo beam breaks) in 30 minutes recording. For PCP vertical and horizontal activity are analysed separately. Data were expressed as the arithmetic mean ±SEM and analysed by ANOVA. In the beam walking test, the time to cross the beam (seconds) and the number of foot slips were expressed as mean ±SEM and analysed by ANOVA and Mann Whitney U test, respectively.

### Results

### Locomotor Activity Test

The spontaneous locomotor activity of rats was measured for 30 minutes placing animals in a novel environmental. The total movement of saline-treated rats 30 minutes post injection was consistent over the studies and corresponded to an average of 400 counts. Morphine sulphate (1, 3 and 10 mg/kg) administered subcutaneously (SC) in naïve rats produced a dose dependent decrease in the spontaneous activity (FIG. 1A; p<0.01). By the MED of 3 mg/kg, the exploratory behaviour of rodents was reduced up to 67% with respect to the controls activity. The highest dose further reduces the movement of naive animals by up to 93% of the spontaneous activity of vehicle-treated rats (28±6 vs 424±23 counts; p<0.01).

The anti-epileptic compound, gabapentin was given orally (PO) at 10, 30 and 100 mg/kg. One-hour post drug administration, gabapentin decreased significantly the locomotor activity of rats at the highest dose only (27% of vehicle-treated group). This effect was significantly different from that induced by morphine 3 mg/kg (262±25 vs 138±26 counts; p<0.01) which consistently reduced the locomotor activity of 61% compared to vehicle treated rats (FIG. 2A).

The psychostimulant substance, PCP was administered at 1 and 10 mg/kg intraperitoneally (ip). At 1 hr post injection both doses increased the horizontal activity in a dose dependent manner (p<0.05) while only the lower dose significantly increased the vertical movement (FIG. 3). Animals treated with 10 mg/kg PCP showed signs of ataxia characterized by lack of paw coordination which was reflected in a decrease in vertical activity (rearing). Although the total activity (vertical+horizontal) did not change significantly in the PCP treated compared to the vehicle-treated group in this experiment (with a standard protocol for the assessment of drug-induced decrease in movement) PCP 10 mg/kg significantly reduced the vertical activity (30±9 vs 186±17 of vehicle-treated group, p<0.01; data not shown) confirming coordination deficits.

### Beam Walking Test

Prior to the assessment of drug-induced motor coordination impairments rats were trained to cross a 75 cm elevated beam and selected based on their performance. Only rats crossing the beam in less than 10 seconds and showing a number of foot slips less than 2 were selected and used for the studies. Usually only 1 rat (sometimes even none) in a group of 40 was found to under perform (<3%). Morphine sulphate was administered at 3 and 10 mg/kg, SC and at 30 minutes, 1, 2 and 3 hours post dosing rats were tested on their ability to cross the elevated beam. The MED (minimal effective dose) in this task was 10 mg/kg and induced a slight increased in the number of foot slips at 30 minutes and 1 hour post drug administration which was not statistically different from controls (1.0±0.6 vs 0.1±0.1 of vehicle treated group at both 30 minutes and 1 hr post dose). The time to cross instead was significantly increased at 30 min post morphine administration only (12.6±0.7 vs 4.1±0.7 of controls; FIG. 3A). The lower dose of 3 mg/kg did not modify the locomotion of rodents in the beam walking at any time point (FIG. 1B). No rats fell off the beam after treatment.

Gabapentin was administered orally at 30, 100 and 300 mg/kg and rats were tested in the beam task at hourly intervals up to 6 hrs (FIG. 2B). Gabapentin produced a dose dependent increased in the number of foot slips starting from the dose of 100 mg/kg. At 1 hour post dose the gabapentin treated group showed an increased in the number of foot slips (1.5±0.5 vs 0.6±0.4 of water treated rats). The peak effect was observed at 2 hours (2.9±0.5 vs 0.2±0.2 of vehicle treated group; **p<0.01) lasting till 4 hours. At 2 and 3 hours 25% of rats fell off the beam. The time to cross was not dramatically modified in gabapentin treated animals and only at 3 and 4 hour post administration the highest dose significantly increase the time to cross (p<0.05).

The effect of a psychostimulant compound was analysed in the beam walking test by testing PCP (0.1-1-10 mg/kg, ip) at 30 minutes, 1, 2, 3 and 4 hour post administration. PCP increased the time taken to cross the beam at the higher dose only while the foot missteps in a dose dependent manner (FIG. 5). Rats treated with 1 mg/kg, showed a slight but significant increase in the number of foot slips at 2 hours post drug administration (p<0.05) while no effect was observed at the lower dose. As expected, rats treated with 10 mg/kg of PCP showed increase in both time to cross and number of footslips. At 30 and 1 hour post administration, 100% of rodents could not even been placed on the beam due to evident lack of paw coordination. At 2 hours, 80% of animals did cross the beam but showing a large number of foot slips (≧5). At this time point, the time taken to cross the beam was still significantly different from controls (p<0.01). At later stages both time and foot slips number decreased and at 4 hrs almost all rats recovered.

### Discussion

In this study we have demonstrated that the beam-walking test is an innovative pre-clinical tool for the assessment of drug-induced dizziness and a component of a comprehensive method for evaluation of the therapeutic index (TI) of new medicines when used in combination with the locomotor activity test. Drug-induced adverse events are often assessed using patient questionnaires in the clinic with data commonly classified in descriptive categories and ranked as percentage or rates. The pre-clinical investigation of such adverse events is complex due to the intrinsic limitations in the animal models. Thus, a number of paradigms have been developed aiming to measure a rodents ability to perform motor tasks (e.g. rota rod or locomotor activity). The interpretation of behaviour data collected are often somewhat confused with the inappropriate use of clinical descriptors such as ataxia or sedation. For example, gabapentin was described as inducing ataxia based on pre-clinical locomotor activity data (Hunter et al, Eur J Pharmacol, 324, 1997) however it is now well established that the main clinical adverse events are dizziness and somnolence and not ataxia (Serpell et al, Pain., 2002, 99: 557-66).

A beam walking task, is commonly used for the assessment of CNS (central nervous system) damage-induced balance and coordination dysfunction (Goldstein and Davies, Beam-walking in rats: studies towards developing an animal model of functional recovery after brain injury, 31, 1990) or for the assessment of motor deficits in genetically modified animals. This paradigm has not been used to examine drug-induced adverse events such as dizziness. Some authors have associated deficits in this task to ethanol-induced ataxia (Crabbe J C et al, Genotypic differences in ethanol sensitivity in two tests of motor incoordination. J Appl Physiol. 2003:1338-51), however ethanol, in humans, induces dizziness, sedation and balance problems as well (Drake C L et al Caffeine reversal of ethanol effects on the multiple sleep latency test, memory, and psychomotor performance, Neuropsychopharmacology. 2003, 28:371-8; Wang G J et al, Regional brain metabolism during alcohol intoxication. Alcohol Clin Exp Res. 2000, 24:822-9). In this study, we demonstrated that the combination of the traditional locomotor activity test with the beam walking task can help to define more precisely the adverse event profile of standard compounds and thus potentially predict the central nervous system (CNS) risk of novel compounds in humans.

Morphine for instance was reported to produce various central adverse events including somnolence, sedation and addiction and we believe that these dominate the adverse effect profile as compared to dizziness (Caldwell J R, Avinza—24-h sustained-release oral morphine therapy. Expert Opin Pharmacother. 2004; 5(2):469-72; Slatkin N E et al, Donepezil in the treatment of opioid-induced sedation: report of six cases. J Pain Symptom Manage. 2001 21 (5):425-38). This is supported pre-clinically by the observation in the locomotor activity test that morphine produced a dose-dependent decrease in both ambulation and rearing with a significant deficit seen at 3 mg/kg. We believe that the effect measured at the lower dose to be due to somnolence rather than dizziness. In fact this dose (3 mg/kg) did not impair the ability of rats to cross the beam and they showed no sign of a deficit in motor coordination. The higher dose of morphine (10 mg/kg), which produced a marked decrease in locomotor activity also impaired performance in the beam-walking task (especially in the time to cross). This is consistent with the sedative-like adverse event of morphine reported in literature (Caldwell J R, Avinza—24-h sustained-release oral morphine therapy. Expert Opin Pharmacother. 2004; 5(2):469-72;).

Gabapentin is an effective medicine used for the treatment of epilepsy and neuropathic pain. Clinical trials have shown that the most frequent adverse events reported were those of dizziness and somnolence (Backonja M et al, Gabapentin for the symptomatic treatment of painful neuropathy in patients with diabetes mellitus: a randomized controlled trial. JAMA, 1998; 280:1831-1836.; Rowbotham M et al, Gabapentin for the treatment of postherpetic neuralgia: a randomized controlled trial. JAMA, 1998; 280:1837-1842). Our studies indicate 100 mg/kg gabapentin in rats produced a small (albeit statistically significant) decrease in locomotor activity. However in the beam walking test this dose produced a robust increase in the number of footslips, whereas the time to cross the beam was not dramatically increased. This is consistent with a deficit in motor coordination and balance i.e. dizziness as opposed to sedation or somnolence.

Phencyclidine (PCP) is a drug, which has been shown to induce ataxia in human (Jacob M S, Phencyclidine ingestion: drug abuse and psychosis Int J. Addict. 1981; Pradhan S N, Phencyclidine (PCP): some human studies Neurosci Biobehav Rev. 1984) and rodents (Melnick et al, A simple procedure for assessing ataxia in rats: effects of phencyclidine, Pharmacol Biochem Behav. 2002). Rats treated with this compound developed observable motor coordination problems, which are reflected in an increase in the number of foot slips in the beam-walking test whilst also increasing activity in the locomotor test.

Based on our observations a significant increase in foot missteps/footslips in the beam-walking test could be clinically related to both ataxia and dizziness. The comparison with the locomotor activity test is therefore important to distinguish between these behaviours. Ataxia is a motor dysfunction defined as inability to co-ordinate muscle activity during voluntary movement (Stedman's Medical dictionary 27^{th} ed). In the locomotor activity data indeed the lack of increase of both vertical and horizontal activity indicates a lack of coordination. This behaviour was only minimally observed with gabapentin and associated with a decrease in ambulation, which indicates a general inactive behaviour (i.e. somnolence).

In conclusion this study supports the claims that the beam walking test is a valuable tool for the assessment of drug-induced dizziness and in combination with other motor tasks, (e.g. locomotor activity test), it can help to improve predictions of the adverse events and therapeutic index of novel compounds.

### FIGURES

FIG. 1: Morphine-induced motor impairments. (A) effect of morphine in the locomotor activity test. Rats were treated with 1, 3 and 10 mg/kg, sc (subcutaneous) and tested 30 minutes post drug administration. (B) effect of morphine in the beam walking test (number of foot slips). Rats were treated with 3 and 10 mg/kg, so and tested at 30 minutes, 1, 2 and 3 hours post administration. A group of animals treated with vehicle were used as negative control in both studies. Data are the mean ±SEM (standard error of the mean) of 8 rats per group. **p<0.01 vs vehicle-treated group (ANOVA) for total counts; NS (not statistically significant group) vs vehicle-treated group (Mann Whitney U test).

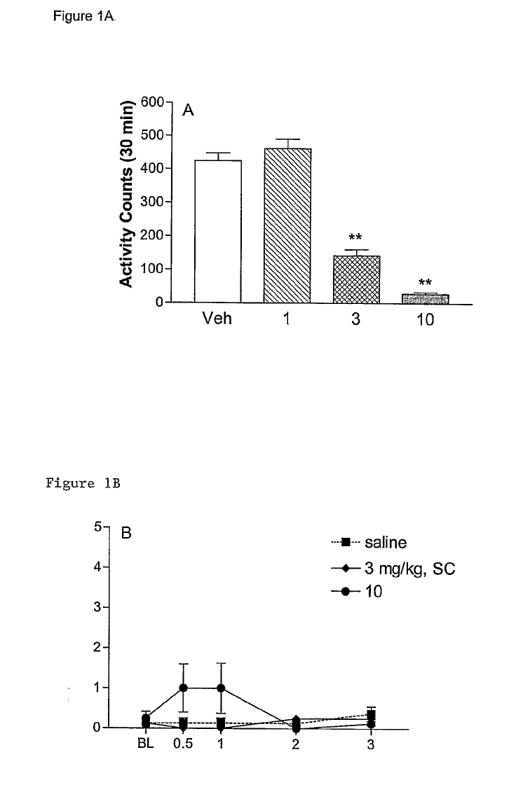

FIG. 2: Gabapentin-induced motor impairments. (A) effect of gabapentin in the locomotor activity test. Rats were treated with 10, 30 and 100 mg/kg, PO and tested 1 hour post drug administration. (B) Effect of morphine in the beam walking test (number of foot slips). Rats were treated with 30, 100 and 300 mg/kg, PO and tested at 30 minutes and every hour up to 6 hours post administration. A group of animals treated with vehicle were used as negative control in both studies. Data are the mean ±SEM of 8 rats per group. *p<0.05 and **p<0.01 vs vehicle-treated group (ANOVA) for total counts; **p<0.01 vs vehicle-treated group (Mann Whitney U test) for foot slips

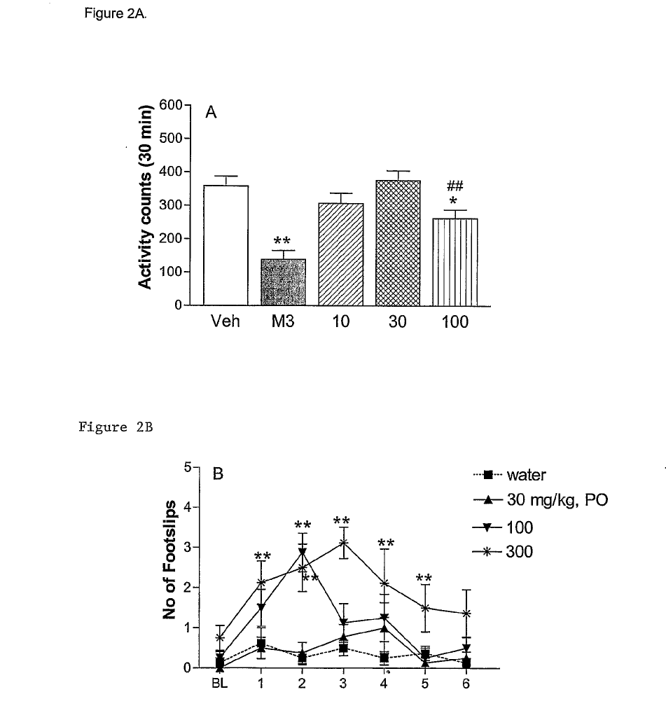

FIG. 3 Morphine (A) and Gabapentin (B) increase time to cross in the beam walking test. Rats were treated with morphine 1, 3 and 10 mg/kg, SC or gabapentin 10, 30 and 100 mg/kg, PO and tested at 30 minutes and every hour up to 4 or 6 hours post administration, respectively. A group of animals treated with vehicle were used as negative control in both studies. Data are the mean ±SEM of 8 rats per group. *p<0.05 and **p<0.01 vs vehicle-treated group (ANOVA) for time to cross.

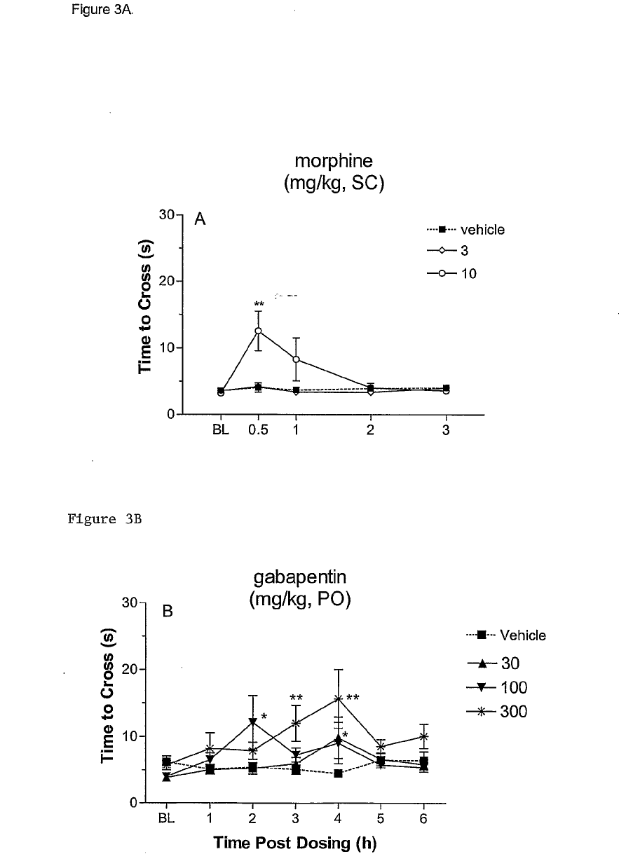

FIG. 4 Phencyclidine (PCP)-induced motor impairments in the locomotor activity test. Rats were treated with PCP (1 and 10 mg/kg, IP [intra-peritoneal]) or vehicle (saline), placed in the locomotor activity cage for acclimatization and tested 1 hour post drug administration. Data are the mean ±SEM of 8 rats per group. *p<0.05 and **p<0.01 vs vehicle-treated group (ANOVA)

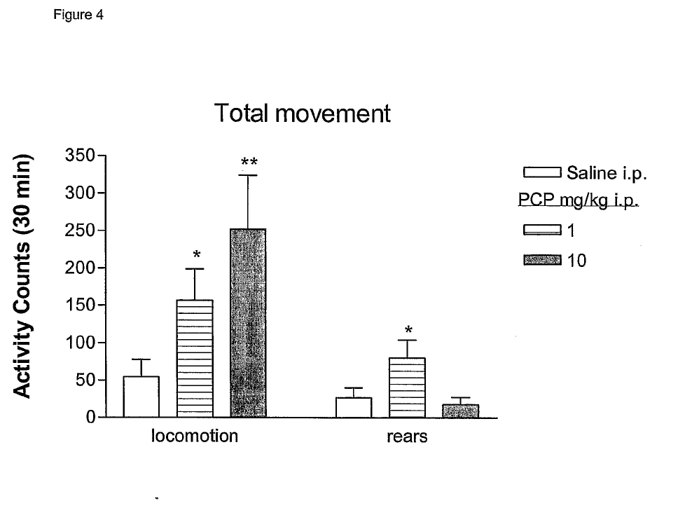

FIG. 5 Phencyclidine (PCP)-induced motor impairments in the beam walking test. Rats were treated with PCP (0.1, 1 and 10 mg/kg, IP) or vehicle (saline) and tested from 30 minutes post dose in the beam walking task. Data are the mean ±SEM of 8 rats per group. **p<0.01 vs vehicle-treated group (ANOVA) for time to cross. *p<0.05 and **p<0.01 vs vehicle-treated group (Mann Whitney U test) for foot slips.

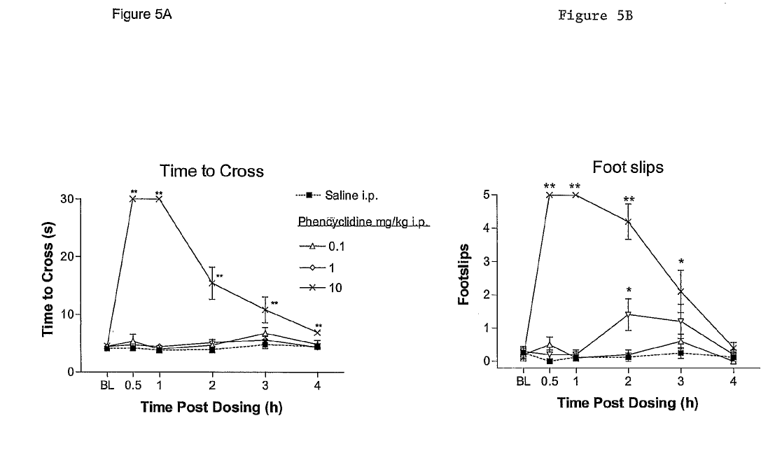

1. A method for determining the degree to which an animal experiences dizziness comprising the following steps:
a) providing a first control animal located on a beam,
b) inducing the control animal to traverse the beam,
c) recording the number of footslips made by the animal during the traversal,
d) providing a second test animal located on the beam or duplicate beam,
e) inducing the test animal to traverse the beam,
f) recording the number of footslips made by the animal during the traversal,
g) determining whether there is an increase, decrease or no change in the number of footslips made by the test animal in comparison to the control animal.

2. A method for assaying a compound for the effect of producing dizziness in animal comprising the following steps:
a) providing a control animal located on a beam,
b) inducing the control animal to traverse the beam,
c) recording the number of footslips made by the animal during the traversal,
d) providing a test animal which has been dosed with a test compound,
e) providing the dosed test animal located on the beam or duplicate beam,
f) inducing the test animal to traverse the beam
g) recording the number of footslips made by the animal during the traversal,
h) determining whether there is an increase, decrease or no change in the number of footslips made by the test animal in comparison to the control animal.

3. The method according to claim 1 further comprising the steps of
a) performing a second different test designed to measure the degree of locomotor activity for the test animal and control animal
b) determining whether there is an increase decrease or no change in the degree of locomotor activity measured for the test animal in comparison to the control animal.

4. The method according to claim 3 wherein the measured locomotor activity is vertical and/or horizontal locomotion.

5. The method according to claim 3 wherein the second test is the locomotor activity test.

6. The method according to claim 1 wherein the control animal and the test animal are the same animal.

7. The method according to claim 1 wherein more than one control and/or test animal are used.

8. The method according to claim 2 further comprising the steps of
a) performing a second different test designed to measure the degree of locomotor activity for the test animal and control animal
b) determining whether there is an increase decrease or no change in the degree of locomotor activity measured for the test animal in comparison to the control animal.

9. The method according to claim 8 wherein the measured locomotor activity is vertical and/or horizontal locomotion.

10. The method according to claim 8 wherein the second test is the locomotor activity test.

11. The method according to claim 2 wherein the control animal and the test animal are the same animal.

12. The method according to claim 2 wherein more than one control and/or test animal are used.
